(19) United States
(12) Patent Application Publication
(10) Pub. No.: US 2024/0382404 A1
(43) Pub. Date: Nov. 21, 2024

(54) SKIN CARE SERUM COMPOSITION WITH NATURAL POLYMERS

(71) Applicant: The Procter & Gamble Company, Cincinnati, OH (US)

(72) Inventors: Lindsey Michelle Britt, Deer Park, OH (US); Shawn David McConaughy, Cincinnati, OH (US)

(21) Appl. No.: 18/667,002

(22) Filed: May 17, 2024

Related U.S. Application Data
(60) Provisional application No. 63/503,052, filed on May 18, 2023.

(51) Int. Cl. A61K 8/73; A61K 8/99; A61Q 19/08

(52) U.S. Cl. CPC A61K 8/731; A61K 8/99; A61Q 19/08; A61K 2800/20; A61K 2800/75

(57) ABSTRACT

A physically stable, uniform, skin care serum composition that contains a polymer system, a skin care active, and a liquid carrier. The polymer system includes microcrystalline cellulose, Sphingomonas ferment extract, and cellulose gum.

### FIELD OF THE INVENTION

The present disclosure relates to a physically stable skin care serum composition containing a natural or naturally derived polymer structurant. More particularly, the serum composition includes a polymer system comprising microcrystalline cellulose, Sphingomonas ferment extract, and cellulose gum.

### BACKGROUND

Serums are popular skin care products that can be packed with skin care actives, like vitamin C, retinol, peptides, and other actives, to quickly deliver on-skin results. Serums are thin-viscosity topical products that generally feel light enough that they can be used under a facial moisturizer, other facial treatments, and/or makeup.

Since serums can have a high concentration of active ingredients, only a few drops are generally needed to deliver results to the full face and/or neck and therefore many serums are dispensed from a dropper package. This package also allows consumers to customize their skin care regimen by adding droplets to another product. Further, the attractive dropper package can suggest effectiveness, as droppers were traditionally used in the pharmaceutical industry, while also connotating luxury and fashion.

Serum products often contain polymers to help maintain physical stability (i.e., no phase separation by visual detection) throughout the shelf life of the product. There are at least two problems with current polymers. First, they often contain ingredients, like polymer structurants, that are not natural or naturally derived and many consumers are demanding skin care products that have biodegradable polymers (in particular, readily biodegradable polymers per OECD 301B) and meet or exceed natural and/or clean beauty standards, such as the requirements to be marketed as “Clean at Sephora” (see Best Clean Beauty Products 2022. Sephora. Retrieved Apr. 19, 2023, from https://www.sephora.com/beauty/clean-beauty-products, “Clean at Sephora” is an initiative in which the beauty retailer badged over 2,000 products as “clean,” in this case meaning free of ingredients like sulfates, parabens, formaldehyde, phthalates, and mineral oil). Second, the synthetic polymers are typically associative polymers that rely on electrostatic interactions as the thickening mechanism, which can catalyze redox reactions with certain skin care actives. Associative polymers also need to be incorporated at a relatively high level for the mechanism to work, which can build too much viscosity, making it difficult to dispense from a dropper and spread a thin layer across a user's face.

It can be difficult to identify a natural polymer that helps maintain physical stability without building too much viscosity and can feel light, silky, and like it quickly absorbs into a user's skin. It is also desirable to identify a natural polymer that can be incorporated into the serum composition using current manufacturing equipment and thus polymer can be incorporated at typical shear rates within current manufacturing capabilities.

Therefore, there is a need for a physically stable skin care serum composition that contains natural polymers that can be dispensed from a dropper.

### SUMMARY

A physically stable, uniform, skin care serum composition, wherein the composition is an emulsion comprising: (a) a continuous phase comprising a polymer system comprising microcrystalline cellulose, Sphingomonas ferment extract, and cellulose gum; a liquid carrier; and a skin care active; (b) a dispersed phase comprising an emollient.

A physically stable, uniform, skin care serum composition comprising: (a) a polymer system comprising microcrystalline cellulose, Sphingomonas ferment extract, and cellulose gum; (b) a liquid carrier comprising water; and (c) a skin care active; and wherein the composition does not comprise an emulsion.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 is a graph showing the viscosity vs. shear rate for different skin care serums;

FIG. 2A is a front view of a dropper including a pipette and a bulb;

FIG. 2B is a front view of a pipette; and

FIG. 3 illustrates a setup for the Tack Method.

### DETAILED DESCRIPTION

It was found that a skin serum composition with a natural or naturally derived polymer system that can include microcrystalline cellulose, Sphingomonas ferment extract, and cellulose gum in combination with a cosmetic carrier and one or more skin care actives can be physically stable and can feel silky during and after application, while also seeming to quickly absorb into skin. Furthermore, serums with microcrystalline cellulose, Sphingomonas ferment extract, and cellulose gum, instead of traditional emulsifiers and synthetic, petroleum-based polymers can have better chemical stability with a variety of common skin care actives including actives (from natural and/or synthetic sources) that are prone to degradation such as vitamin E (tocopherol) and its derivatives, vitamin C and its derivatives, niacinamide, p-coumaric acid, retinol and its derivatives, botanicals, or combinations thereof. An added benefit of the polymer system is that some of the ingredients, such as Sphingomonas ferment extract (a healthy postbiotic), can be anti-inflammatory and can help soothe irritation.

The skin serum composition can be an emulsion, or it can be a serum with a single phase that is free of, substantially free of, or formulated without oil and/or silicones (i.e., water-based serum). The composition can be physically phase stable. A composition is physically stable if by visual detection there is no phase separation. As used herein, “visual detection” means that a human viewer can visually discern if the product had separated into 2 distinct layers in a 2 oz clear glass jar with the unaided eye (except for standard corrective lenses adapted to compensate for near-sightedness, farsightedness, astigmatism, and/or other corrected vision) in lighting at least equal to the illumination of a standard 100-watt incandescent white light bulb at 30 cm.

When skin care serums are emulsions (e.g., oil-in-water emulsions) they generally require emulsifiers to promote the formation of the emulsion and help maintain product stability. However, some emulsifiers have the potential to cause skin irritation, are not natural or naturally derived, and/or do not satisfy the “Clean at Sephora” guidelines and therefore may not be preferred by some consumers. It can also be difficult to identify emulsifiers that are compatible with the polymers in the serum compositions.

It was found that a polymer system comprising microcrystalline cellulose, Sphingomonas ferment extract, and cellulose gum could also act as an emulsifier and in some instances, it could eliminate the need for one or more additional emulsifiers in the formula. Limiting or eliminating additional emulsifiers can be desirable for consumers who seek products with a limited number of ingredients, to limit skin irritation, and it can also make the product more efficient by limiting the ingredients that provide limited or no benefit to a user's skin. In one example, the composition is formulated without, free of, or substantially free of traditional emulsifiers other than those in the polymer system.

### Skin Care Composition

Many consumers prefer a serum product that has sufficient zero shear viscosity that if the user applies droplets directly to her face they do not quickly run down their face. However, when shear is applied, generally by the consumer rubbing the serum into their face with their fingertips or palms, the serum easily spreads into a thin, uniform layer, that can be quickly absorbed. It was found that compositions containing microcrystalline cellulose, Sphingomonas ferment extract, and cellulose gum can have a unique, consumer preferred rheology profile that can provide several benefits that are noticeable to consumers.

In some examples, the serum composition can have a pseudoplastic rheology across certain shear rates. More particularly, a curve fit of the rheology profile can be determined using the Carreau Yasuda Model, which is an empirical equation that fits non-Newtonian data.

A physically phase stable, uniform, serum composition can have the following rheology values, as measured according to the Rheology Test, described hereafter:
At 0.01 s⁻¹ shear rate, the viscosity can be less than or equal to 300 Pa·s
At 20 s⁻¹ shear rate the viscosity can be less than or equal to 0.6 Pa·s
Between 0.01 s⁻¹ and 0.1 s⁻¹ shear rate, the slope (with viscosity on the y-axis) is greater than −600 and less than 0. The slope is calculated by measuring the viscosity at 0.1 s⁻¹ and 0.1 s⁻¹ and using the following equation:

$$slope = \frac{viscosity at 0.1 {s}^{- 1} - viscosity at 0.01 {s}^{- 1}}{0.1 {s}^{- 1} - 0.01 {s}^{- 1}}$$

The unique rheology profile can help deliver at least the following benefits:
The viscosity at 0.01 s⁻¹ shear rate, indicates that the serum can have a viscous silky texture as the consumer begins to spread it across their face with their fingertips.
The slope between 0.01 s⁻¹ and 0.1 s⁻¹ shear rate, indicates that the serum can have sufficient viscosity, so the user continues to feel the composition during application.
The low viscosity at 20 s⁻¹ shear rate indicates that the serum can feel like it has absorbed and/or is weightless and the skin can feel smooth, as the user finishes spreading the serum over their face.
The serum composition can be dispensed through a standard dropper for skin care products because squeezing the dropper causes high shear, which easily dispenses a droplet of serum. Some current products have too much viscosity and are not as shear thinning, which can require a larger dropper to dispense a droplet. A larger dropper is not consumer preferred because with serum composition consumers like to be able to tightly control the dosage and they may also believe that large droplets are wasteful.

The skin care composition can be a serum composition with a relatively low viscosity. The viscosity at 0.01 s⁻¹ shear rate can be less than or equal to 700 Pa·s, alternatively less than or equal to 500 Pa·s, alternatively less than or equal to 400 Pa·s, and alternatively less than or equal to 300 Pa·s, according to the Rheology Test Method, described hereafter.

Tack force generally refers to the peak force required to separate an object from the composition after contacting the composition. An increase in tack force is typically accompanied by an increase in stringiness and the serum feels heavy and sticky on top of the user's skin. Instead, serum users want the product to feel weightless, silky, and smooth. The time weighted force area can help predict the moisturization signal perceived by a user. A lower time weighted force area, according to the Tack Method, described hereafter, can indicate that the serum composition may be more preferred by users as compared to compositions with a higher time weighted force area.

The serum compositions can have a time weighted force area of between about 1×10⁴ g to about 2.2×10⁴ g, alternatively from about 1.1×10⁴ g to about 2.0×10⁴ g, alternatively from about 1.2×10⁴ g to about 1.9×10⁴ g, alternatively from about 1.3×10⁴ g to about 1.7×10⁴ g, according to the Tack Method, described hereafter. The serum compositions can have a time weighted force area of less than 2.3×10⁴ g, alternatively less than 2.1×10⁴ g, alternatively less than 2.0×10⁴ g, alternatively less than 1.9×10⁴ g, alternatively less than 1.8×10⁴ g, and alternatively less than 1.7×10⁴ g, according to the Tack Method, described hereafter.

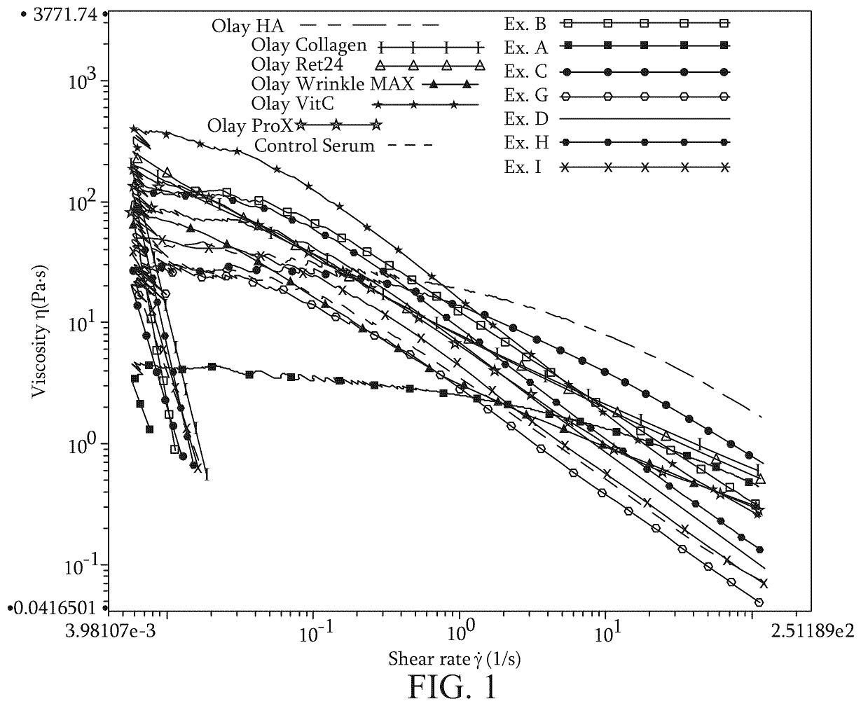

FIG. 1 shows the rheology curve, viscosity vs. shear rate, for compositions with a polymer system containing microcrystalline cellulose, Sphingomonas ferment extract, and cellulose gum (“inventive examples”) as compared to current in-market products and other comparative example formulas, as described in Table 1, Table 2, Example D in Table 4, and Table 5 to Table 7, described hereafter. FIG. 1 shows that the inventive examples had a rheology profile that was about the same or better than current commercially available products. In particular, the graphs show the inventive examples have a relatively high viscosity at a low shear rate and as the shear increases, the viscosity decreases until it is very low at the highest shear rates tested.

In some examples, the composition can have a limited number of ingredients, in particular a limited number of ingredients that need to be reported on the INCI (International Nomenclature Cosmetic Ingredient) statement on the package. The composition can be ≤25 ingredients, alternatively ≤20 ingredients, alternatively ≤15 ingredients, and alternatively ≤12 ingredients.

### Polymer System

The polymer system can contain Microcrystalline cellulose, Sphingomonas ferment extract, and cellulose gum. This combination is available as PemuPur™ START Polymer by Lubrizol®, which is readily biodegradable according to OECD 301F and natural (96% NOC (naturally occurring carbon) according to ISO16128).

PemuPur™ is sold as a powder and the inventors found that this material can be incorporated into consumer preferred serum compositions. The Sphingomonas ferment extract and cellulose gum can be partially solubilized by the continuous water phase. The microcrystalline cellulose can be uniformly dispersed throughout the continuous phase and/or the liquid carrier.

When the serum composition is made, the PemuPur™ can be milled, as described in the Finetuned High Shear Method, hereafter. If the particles, including microcrystalline cellulose aggregates, are too large, they can be visible in the serum product and/or the product can feel gritty, neither of which is consumer preferred. The serum composition can have no visible particles, including no visible microcrystalline cellulose particles, as determined by visual detection. Typically, microcrystalline cellulose is in the form of particulate aggregates, having an average particle diameter of from about 5 μm to about 20 μm, alternatively from about 10 μm to about 20 μm, alternatively from about 10 μm to about 25 μm, alternatively from about 10 μm to about 30 μm, alternatively from about 10 μm to about 40 μm, alternatively from about 10 μm to about 50 μm as determined using a Keyence™ VHX-7100 microscope and the accompanying image analysis hardware/software provided with it, updated as of May 18, 2023.

The skin care serum composition can contain a weight ratio of microcrystalline cellulose to Sphingomonas ferment extract that is greater than or equal to 1:1, alternatively it can be from about 1:2 to about 4:1, alternatively it can be from about 2:1 to about 4:1. The skin care serum composition can contain a weight ratio of Sphingomonas ferment extract to cellulose gum that is greater than or equal to 1:1. The skin care serum composition can contain a weight ratio of microcrystalline cellulose to cellulose gum that is greater than or equal to 1:1, alternatively it can be from about 10:1 to about 2:1, alternatively from about 9:1 to about 3:1, alternatively from about 5:1 to about 3:1. The polymer system can contain from about 20% to about 90%, alternatively about 30% to about 75%, alternatively about 40% to about 70%, by weight of the polymer system, microcrystalline cellulose. The polymer system can contain from about 10% to about 80%, alternatively from about 20% to about 75%, alternatively from about 25% to about 45%, alternatively from about 25% to about 35%, by weight of the polymer system, of Sphingomonas ferment extract. The polymer system can contain from greater than 0% to about 20%, alternatively from about 5% to about 20%, alternatively from about 5 to about 15%, alternatively from about 8% to about 12%, by weight of the polymer system, of cellulose gum.

The composition can contain less than 1%, alternatively less than 0.9%, alternatively less than 0.8% of a polymer system comprising microcrystalline cellulose, Sphingomonas ferment extract, and cellulose gum. The composition can contain more than 0.5%, alternatively more than 0.6% of a polymer system comprising microcrystalline cellulose, Sphingomonas ferment extract, and cellulose gum. The composition can contain from about 0.50% to about 0.95%, alternatively from about 0.55% to about 0.9%, alternatively from about 0.60% to about 0.85%, alternatively from about 0.62% to about 0.85%, alternatively from about 0.65% to about 0.80%, and alternatively from about 0.67% to about 0.75% of a polymer system comprising microcrystalline cellulose, Sphingomonas ferment extract, and cellulose gum.

The polymer system can be formulated without, free from, or substantially free from xanthan gum, hydroxyethylcellulose, and combinations thereof. The polymer system can be formulated without, free from, or substantially free from synthetic polymers. The polymer system can be formulated without, free from, or substantially free from carbomers, acrylates/C10-30 alkyl acrylate crosspolymer (commercially available as Pemulen™ TR-1 polymer from Lubrizol®), polyacrylamide, sodium acrylate/sodium acryloyldimethyl taurate copolymer, hydroxyethyl acrylate/sodium acryloyldimethyl taurate copolymer, polyacrylate crosspolymer-6, acrylates copolymer, ammonium acryloyldimethyltaurate, or combinations thereof.

### Skin Care Actives

It was found that serums with a polymer system comprising microcrystalline cellulose, Sphingomonas ferment extract, and cellulose gum, instead of traditional emulsifiers and synthetic, petroleum-based polymers can be combined with a variety of skin care actives. The polymer system can have better chemical stability with a variety of common skin care actives including actives that are prone to degradation including vitamin E (tocopherol), vitamin C, retinol, undecylenoyl phenylalanine, botanicals, pre-, pro- or post-biotics, and/or derivatives and mixtures thereof.

Non-limiting examples of botanicals can include carnosic acid and rosmarinic acid from rosemary, Curcuma longa root extract, green tea leaf extract carica papaya fruit extract, colloidal oatmeal, Chrysanthemum parthenium extract, Glycyrrhiza glabra root extract, Cichorium intybus root extract, Melia azadirachta Leaf Extract, Melia azadirachta flower extract, Corallina officinalis extract, Ocimum basilicum flower/leaf extract, Ocimum sanctum leaf extract, Vaccinium vitis-idaea fruit extract, Citrus unshiu peel extract, Hippophae rhamnoides fruit extract, Litchi chinensis seed extract, Mangifera indica leaf extract, and/or derivatives and mixtures thereof. Botanicals can include extracts and purified naturally occurring compounds.

Non-limiting examples of pre-, pro-, and post-biotics can include actives chosen from Galactomyces ferment filtrate, inositol, Lactococcus ferment lysate, Lactobacillus, and combinations thereof.

If the skin care composition is an emulsion, the one or more skin care active can be in the continuous phase and/or the dispersed phase.

There are a large number of skin care actives that can improve the health and/or physical appearance of a user's skin. For example, retinoids are another example of skin care actives used in skin care compositions to reduce signs of aging skin.

Non-limiting examples of skin care actives can include naturally occurring and/or synthetic peptides (e.g., di-, tri-, tetra-, and penta-peptides and derivatives); essential oils, skin sensates, astringents (e.g., clove oil, menthol, camphor, eucalyptus oil, eugenol, menthyl lactate, witch hazel distillate); antimicrobial agents (e.g., iodopropyl butylcarbamate), antioxidants, skin brightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives ethyl panthenol, aloe vera, pantothenic acid and its derivatives, allantoin, bisabolol, and dipotassium glycyrrhizinate); farnesol, phytantriol, anti-acne actives (e.g., resorcinol, sulfur, salicylic acid, benzoyl peroxide, erythromycin, and zinc); hydroxy acids (e.g., alpha-hydroxy acids such as lactic acid and glycolic acid or beta-hydroxy acids such as salicylic acid and salicylic acid derivatives such as the octanoyl derivative); phytic acid; lipoic acid; lysophosphatidic acid, vitamin B₃ compounds including niacinamide; retinoids; ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox®), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, rosemary extracts, sunscreen actives.

### Vitamin B₃ Compound

The compositions herein can include a safe and effective amount of a vitamin B₃compound. The Vitamin B₃ compound can be in the continuous phase. In some instances, the present compositions may contain 0.01% to 10%, by weight, of the vitamin B₃ compound, based on the weight or volume of the composition (e.g., 0.1% to 10%, 0.5% to 5%, or even 1% to 3%). As used herein, “vitamin B₃ compound” means a compound having the formula:

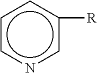

Where: R is CONH₂ (i.e., niacinamide), COOH (i.e., nicotinic acid) or CH₂OH (i.e., nicotinyl alcohol); derivatives thereof; and salts of any of the foregoing.

Exemplary derivatives of vitamin B3 compounds include nicotinic acid esters, including non-vasodilating esters of nicotinic acid (e.g., tocopheryl nicotinate, myristyl nicotinate) nicotinamide riboside, nicotinyl amino acids, nicotinyl alcohol esters of carboxylic acids, nicotinic acid N-oxide, and niacinamide N-oxide.

### Retinoid

The skin care compositions herein can include a safe and effective amount of a retinoid and the retinoid can be in the dispersed phase of an emulsion. As used herein, “retinoid” includes all natural and/or synthetic analogs of Vitamin A or retinol or retinol-like compounds which possess the biological activity of Vitamin A in the skin, as well as the geometric isomers and stereoisomers of these compounds. For example, the retinoid may be a retinol ester (e.g., C2-C22 alkyl esters of retinol, including retinyl palmitate, retinyl acetate, and retinyl propionate), retinol aldehydes, retinal, beta-carotene, and/or retinoic acid (including all-trans retinoic acid and/or 13-cis-retinoic acid). A particularly suitable example of a retinoid for use in the present composition is retinyl propionate (“RP”). These compounds are well known in the art and are commercially available from a number of sources, e.g., Sigma Chemical Company (St. Louis, MO), Boerhinger Mannheim (Indianapolis, IN), BASF® (Mt. Olive, NJ), and Roche (Basel, Switzerland). Other suitable retinoids are tocopheryl-retinoate (tocopherol ester of retinoic acid (trans- or cis-), adapalene {6-[3-(1-adamantyl)-4-methoxyphenyl]-2-naphthoic acid}, and tazarotene (ethyl 6-[2-(4,4-dimethylthiochroman-6-yl)-ethynyl]-nicotinate). The retinoid may be included as a pure or substantially pure material, or as an extract obtained by suitable physical and/or chemical isolation from natural (e.g., plant) sources.

The serum composition may contain 0.0001% to 2% (e.g., 0.005% to 2%, 0.01% to 1%, 0.01% to 0.5%, 0.1% to 0.4%, 0.15% to 0.3%) of the retinoid. In some instances, mixtures of more than one retinoid may be used.

The retinoid present in the serum compositions herein can be stable. For the skin care composition to provide the desired health or appearance benefit, it is important to provide a suitable amount of retinoid active.

The composition can include retinyl propionate (RP) and can have ≥90% RP remaining, ≥alternatively 85% RP remaining, alternatively ≥80% RP remaining, alternatively ≥75% RP remaining, alternatively ≥70% RP remaining, alternatively ≥65% RP remaining, alternatively ≥60% RP remaining, alternatively ≥55% RP remaining, alternatively ≥50% RP remaining at any time during the shelf life of the product. The % RP remaining is determined using the HPLC Method, described hereafter. The initial RP is the concentration of RP at the time of manufacture, which can be the % RP claimed on the product packaging or other materials (e.g., website, SmartLabel®, etc.).

It was found that a relatively high level of sodium hyaluronate (HA) (e.g., 0.5% or 1%) with a molecular weight between 500 kDa and 1000 kDa in combination with a polymer system comprising microcrystalline cellulose, Sphingomonas ferment extract, and cellulose gum can build too much viscosity, leading to serums that are difficult to dispense and have a tacky, stringy texture. Furthermore, it was found that the compositions containing 1% HA and 1% polymer system and the compositions containing 0.5% HA and 0.5% polymer system were physically unstable and phase separation was visually discerned, though this happened more quickly with the higher HA and polymer levels of 1% each. Thus, in some examples, the serum composition is formulated without, frec of, or substantially free of HA. In some examples, the scrum composition is formulated without, free of, or substantially free of HA with a molecular weight of greater than or equal to 500 kDa, alternatively 750 kDa, alternatively 1000 kDa. If the skin care composition is an emulsion and the skin care actives, in particular natural skin care actives, are a different color than the rest of the formula (e.g., rosemary or green tea extract), it can be desirable for the skin care active to be in the continuous phase, so the product appears more uniform in the event of any dispersed phase droplet coalescence.

In some examples, the compositions contain HA and can contain less than 0.5%, alternatively less than 0.4% HA, alternatively less than 0.3% HA, alternatively less than 0.2% HA, alternatively less than 0.1% HA, and alternatively less than or equal to 0.05% HA.

In some examples, the composition can be free of or substantially free of snow mushroom (Tremella fuciformis) extract. In other examples, the composition can contain snow mushroom extract and can have less than 0.5%, alternatively less than 0.3%, and alternatively less than or equal to 0.1% snow mushroom. It was found that a serum containing 1% snow mushroom polysaccharide (supplied by Shanghai Huiwen Biotech Corp., Ltd.) and a polymer system comprising microcrystalline cellulose, Sphingomonas ferment extract, and cellulose gum was not a smooth consistency, instead it had a consistency similar to cottage cheese.

### Emulsifier

In some examples, the skin care composition is free of an emulsifier other than the ingredients in the polymer system.

In other examples, the skin care composition can contain an emulsifier in addition to the ingredients in the polymer system. The emulsifier can promote the formation of the emulsion, stabilize the composition, and can be chemically and physically compatible with the other components of the composition.

The skin care composition can contain from about 0.10% to about 2%, alternatively from about 0.2% to about 1.5%, alternatively from about 0.4% to about 1.2%, and alternatively from about 0.6% to about 0.8% of an emulsifier. The skin care composition can contain less than 2%, alternatively less than 1.75%, alternatively less than 1.5%, alternatively less than 1.25%, alternatively less than 1%, and alternatively less than 0.8% of an emulsifier.

The emulsifier can be a non-silicone containing emulsifier. Non-limiting examples of non-silicone containing emulsifiers can include polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, polyoxyethylene 20 sorbitan trioleate (Polysorbate 85), sorbitan monolaurate, polyoxyethylene 4 lauryl ether sodium stearate, polyglyceryl-4 isostearate, polyglyceryl-3 laurate, sodium stearoyl glutamate, hexyl laurate, PPG-2 methyl glucose ether distearate, PEG-100 stearate, cetearyl glucoside and mixtures thereof.

The emulsifier can be a fatty acid ester mixtures based on a mixture of sorbitan or sorbitol fatty acid ester and sucrose fatty acid ester, wherein the fatty acid in each case is preferably C₈-C₂₄, more preferably C₁₀-C₂₀. The preferred fatty acid ester emulsifier is a mixture of sorbitan or sorbitol C₁₆-C₂₀ fatty acid esters with sucrose C₁₀-C₁₆ fatty acid esters, especially sorbitan stearate and sucrose cocoate. This is commercially available from ICI under the trade name Arlatone 2121.

The skin care composition can include a silicone-containing emulsifier. A wide variety of silicone containing emulsifiers can be used. These silicone emulsifiers are typically organically modified organopolysiloxanes which are also known to those skilled in the art as silicone surfactants. Useful silicone emulsifiers include dimethicone copolyols. These materials are polydimethylsiloxanes which have been modified to contain polyether side chains such as polyethylene oxide chains, polypropylene oxide chains, mixtures of these chains, and polyether chains containing groups derived from both ethylene oxide and propylene oxide. Other examples include alkyl-modified dimethicone copolyols, that is, compounds containing pendant C₂-C₃₀ side chains.

Dimethicone copolyols can also be used as an emulsifiers herein including polydimethylsiloxane polyether copolymers with pendant polyethylene oxide sidechains, polydimethylsiloxane polyether copolymers with pendant polypropylene oxide sidechains, polydimethylsiloxane polyether copolymers with pendant mixed polyethylene oxide and polypropylene oxide sidechains, polydimethylsiloxane polyether copolymers with pendant mixed poly (ethylene) (propylene) oxide sidechains, polydimethylsiloxane polyether copolymers with pendant organobetaine sidechains, polydimethylsiloxane polyether copolymers with pendant carboxylate sidechains, polydimethylsiloxane polyether copolymers with pendant quaternary ammonium sidechains; and also further modifications of the preceding copolymers containing pendant C2-C30 straight, branched, or cyclic alkyl moieties. Examples of commercially available dimethicone copolyols useful herein sold by Dow Corning Corporation are Dow Corning® 190, 193, Q2-5220, 2501 Wax, 2-5324 fluid, and 3225C (this later material being sold as a mixture with cyclomethicone). Cetyl dimethicone copolyol is commercially available as a mixture with polyglyceryl-4 isostearate (and) hexyl laurate and is sold under the tradename ABIL® WE-09 (available from Goldschmidt). Cetyl dimethicone copolyol is also commercially available as a mixture with hexyl laurate (and) polyglyceryl-3 oleate (and) cetyl dimethicone and is sold under the tradename ABIL® WS-08 (also available from Goldschmidt). Other nonlimiting examples of dimethicone copolyols also include lauryl dimethicone copolyol, dimethicone copolyol acetate, dimethicone copolyol adipate, dimethicone copolyolamine, dimethicone copolyol behenate, dimethicone copolyol butyl ether, dimethicone copolyol hydroxy stearate, dimethicone copolyol isostearate, dimethicone copolyol laurate, dimethicone copolyol methyl ether, dimethicone copolyol phosphate, and dimethicone copolyol stearate. See International Cosmetic Ingredient Dictionary, Fifth Edition, 1993, which is incorporated by reference herein in its entirety.

In another example, the emulsifier can include cetearyl olivate and sorbitan olivate (commercially available as Olivem® 1000 from HallStar®) and/or polyglyceryl-3 cetearyl ether olivate (commercially available as Galolive® Bioten from Acme-Hardesty®). It is believed that other emulsifiers that include a mixture of waxy esters made from fatty alcohols and fatty acids derived from natural sources (e.g., olive, palm, corn, palm, rapeseed, soybean, sunflower, coconut, tallow, canola, castor) with a carbon chain length ranging from C12-C22 could be used in stable skin care compositions.

Additional examples of emulsifiers that can be used are found in U.S. Pat. No. 6,174,533, incorporated herein by reference.

### Emollient

If the composition is an emulsion, the composition may include one or more emollients in the dispersed phase. One or more emollients can be natural or naturally derived and/or biodegradable, alternatively all the emollients can be natural or naturally derived and/or biodegradable. The skin care composition can be formulated without, free of, or substantially free of petroleum-based emollients. The emollient can be a liquid emollient having a melting point below 40° C., below 35° C., or below 30° C. The liquid emollient can be an oil, which can include an ester, alkane, triglyceride, non-volatile silicones, and combinations thereof. The emollient can be a mono- and/or poly-unsaturated oil. In another example, the emollient can be a waxy emollient, such as a fatty alcohol, having a melting point above 40° C., alternatively above 50° C. In some examples, the skin care composition can contain one or more liquid emollients and one more waxy emollients.

The skin care composition can include from about 0.5% to about 20%, alternatively from about 1% to about 17%, alternatively from about 2% to about 15%, alternatively from about 3% to about 12% emollient, alternatively from about 4% to about 10%, and alternatively from about 5% to about 8% of one or more emollients.

### Liquid Emollient

The skin care composition may include from about 0.5% to about 15%, alternatively from about 0.7% to about 12%, alternatively from about 0.8% to about 10%, alternatively from about 1% to about 9%, alternatively from about 1% to about 8%, alternatively from about 2% to about 7%, alternatively from about 3% to about 6% liquid emollient.

The liquid emollient can be a naturally derived oil that is a plant oil. Examples of the plant oil can include, but are not limited to, palm kernel, coconut, avocado, canola, corn, cottonseed, olive, palm, hi-oleic sunflower, mid-oleic sunflower, sunflower, palm stearin, palm kernel olein, safflower, babassu oils, and combinations thereof. In one embodiment, palm kernel oil may be the selected oil. In another embodiment, coconut oil may be the selected oil. In another embodiment, the plant oil may be a combination of palm kernel oil and coconut oil.

The skin care composition can include one or more of the following liquid emollients:
An alkane or isoalkane, ranging in total carbon from about C8-C60, including but not limited to squalane, squalene, coconut alkanes, coconut/palm kernel alkanes, C9-12Alkane, C10-13 Alkane, C13-15 Alkane, C15-19 Alkane, C14-22 Alkane, C30-34 Alkane, C9-20 Isoalkane, C10-12 Isoalkane, C12-15 Isoalkane, isohexadecene, C32-54 Isoalkane, or a combination thereof.
An ester formed from an alcohol having from 1-6 carbons and acid having from about 6-22 carbons, including but not limited to isopropyl isostearate, isopropyl palmitate, isoamyl laurate, shea butter ethyl esters, shea butter cetyl esters, shea butter decyl esters, shea butter oleyl esters, jojoba esters, coco-capylate/caprate, ethyl caprate, ethylhexyl caprate, isoamyl caprylate/caprate, argan oil isostearyl esters, babassu seed oil ethyl esters, camelina sative seed oil ethyl esters, castor/olive oil esters, coconut oil decyl esters, coconut oil ethyl esters, linseed oil ethyl esters, olive oil decyl esters, olive oil ethylhexyl esters, palm kernel oil triacetin esters, palmitoyl serine/silk amino acid methyl esters, rapeseed oil decyl esters, safflower seed oil decyl esters, sunflower seed oil cetyl esters, sunflower seed oil ethyl esters, trehalose isostearate esters, or a combination thereof.
A triglyceride having 3 carbon chains where the carbon chain length ranges from about C7-C18, including but not limited to triheptanoin, caprylic/capric triglycride, C10-19triglycerides, caprylic/capric/lauric triglycerides, hydrogenated C12-C18 triglycerides, triethylhexanoin, or a combination thereof.
A non-volatile silicone fluid including but not limited to dimethicone, amodimethicone, or a combination thereof.

### Waxy Emollient

The skin care composition may include from about 0.1% to about 1.5%, alternatively from about 0.2% to about 1.2%, and alternatively from about 0.5% to about 1%.

One or more emollients can be solids at room temperature and have an average carbon chain length ranging from about 12 to about 22, alternatively about 12 to about 18, alternatively from about 16 to about 22, and alternatively 16 to about 18.

The skin care composition can include a waxy emollient, such as a wax, butter, fatty alcohol, or a combination thereof. Waxy emollients can include, but are not limited to, beeswax, jojoba wax, shea butter, palm kernel wax, lanolin wax, shellac wax, rice bran wax, carnauba wax, ozokerite, cocos nucifera (coconut) butter, goat butter, acrocomia aculeata seed butter, chiuri butter, carnauba, candelilla, or a combination thereof.

Fatty alcohols can include monohydric alcohols having 8-22 carbon atoms, although longer chain alcohols in excess of 30 carbons may be used. The fatty alcohols may be saturated or unsaturated. The fatty alcohols may be straight or branched. In one example, the waxy emollient may comprise straight chain, saturated fatty alcohol with a terminal hydroxyl. The fatty alcohols can include decyl alcohol, lauryl alcohol, myristyl alcohol, cetyl alcohol, stearyl alcohol, isostearyl alcohol, icosyl alcohol, behenyl alcohol, batyl alcohol, arachidyl alcohol, brassica alcohol, coconut alcohol, hydrogenated jojoba alcohol, jojoba alcohol, or a combination thereof. In some examples, the fatty alcohol can have an average carbon chain length from about 12 to about 22, alternatively from about 16 to about 22, alternatively from about 16 to about 18. Alternatively, the fatty alcohol can have an average chain length of from about 12 to about 18. In some examples, the skin care compositions can be formulated without, substantially free of, or free of cetearyl alcohol because they believe that it can have unfavorable comedogenicity. In another example, the skin care compositions can be formulated without, substantially free of, or free of palmitic acid and stearic acid.

### Dermatologically Acceptable Carrier

The skin care composition can include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 95%, about 52% to about 90%, about 55% to about 85%, or, alternatively, from about 60% to about 80%, by weight of the composition.

The carrier can be in a wide variety of forms. In some instances, the solubility or dispersibility of the components (e.g., extracts, sunscreen active, additional components) may dictate the form and character of the carrier. Non-limiting examples of the product form can include dispersions, emulsions, and solid forms (e.g., gels, sticks, flowable solids, or amorphous materials). The skin care composition can be an aqueous single-phase or an emulsion. The oil phase of the emulsion may include silicone oils, esters, ethers, triglcyerides and mixtures thereof. The aqueous phase may include water and water-soluble ingredients (e.g., water-soluble moisturizing agents, conditioning agents, anti-microbials, humectants and/or other skin care actives). In some instances, the aqueous phase may include components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s). The composition can contain from about 1% to about 20%, alternatively from about 3% to about 18%, alternatively from about 5% to about 17%, and alternatively from about 6% to about 15% humectant.

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more non-volatile oils (e.g., botanical oils and/or silicone oils). Some nonlimiting examples of oils that may be suitable for use in the present compositions are disclosed in U.S. Pat. No. 9,446,265 and U.S. Publication No. 2015/0196464.

The carrier may contain one or more dermatologically acceptable diluents. As used herein, “diluent” refers to materials in which the skin care actives herein can be dispersed, dissolved, or otherwise incorporated. Some non-limiting examples of hydrophilic diluents include water, organic hydrophilic diluents such as lower monovalent alcohols (e.g., C₁-C₄) and low molecular weight glycols and polyols, glycerol, butylene glycol, 1,2,4-butanetriol, sorbitol esters, 1,2,6-hexanetriol, ethanol, isopropanol, sorbitol esters, butanediol, ether propanol, and combinations thereof.

### Other Optional Ingredients

The present composition may optionally include one or more additional ingredients commonly used in cosmetic compositions (e.g., dyes, pigments, mica, insoluble particles, skin care actives, anti-inflammatory agents, sunscreen agents, emulsifiers, buffers, rheology modifiers, chelants, combinations of these and the like), provided that the additional ingredients do not undesirably alter the skin health or appearance benefits provided by the present compositions. The additional ingredients can be in the continuous phase and/or the dispersed phase. The additional ingredients, when incorporated into the composition, should be suitable for use in contact with human skin tissue without undue toxicity, incompatibility, instability, allergic response, and the like. Some nonlimiting examples of additional actives include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunless tanning agents, lubricants, anti-acne actives, anti-cellulite actives, chelating agents, anti-wrinkle actives, anti-atrophy actives, phytosterols and/or plant hormones, N-acyl amino acid compounds, antimicrobials, and antifungals. In some examples, the composition can include a fragrance, in particular a natural fragrance, or a colorant, in particular a natural colorant. The chelant can be selected from ethylenediaminetetraacetic acid (EDTA), trisodium ethylenediamine disuccinate (EDDS), trisodium dicarboxymethyl alaninate, tetrasodium glutamate diacetate, phytic acid, or a combination thereof. Other non-limiting examples of additional ingredients and/or skin care actives that may be suitable for use herein are described in U.S. Publication Nos. 2002/0022040; 2003/0049212; 2004/0175347; 2006/0275237;2007/0196344; 2008/0181956; 2008/0206373; 2010/00092408; 2008/0206373; 2010/0239510;

2010/0189669; 2010/0272667; 2011/0262025; 2011/0097286; US2012/0197016; 2012/0128683;2012/0148515; 2012/0156146; and 2013/0022557; and U.S. Pat. Nos. 5,939,082; 5,872,112;6,492,326; 6,696,049; 6,524,598; 5,972,359; and 6,174,533.

In some examples, the composition can contain less than 10% silicone elastomer, alternatively less than 7%, alternatively less than 5%, alternatively less than 3%, alternatively less than 1%, alternatively the composition can be formulated without, free of, or substantially free of silicone elastomer. It was found that serum compositions that included 10% silicone elastomer and a polymer system containing microcrystalline cellulose, Sphingomonas ferment extract, and cellulose gum were not physically stable.

When including optional ingredients in the compositions herein, it may be desirable to select ingredients that do not form complexes or otherwise undesirably interact with other ingredients in the composition, especially pH sensitive ingredients like niacinamide, salicylates and peptides. When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition.

In some examples, the composition can be formulated without, substantially free of, or free of dyes.

### Method of Use

The skin care serum composition may be applied to the face, neck, and/or a portion or combination thereof at least once a day, twice a day, or on a more frequent daily basis, during a treatment period. When applied twice daily, the first and second applications are separated by at least 1 to 12 hours. Typically, the composition is applied in the morning and/or at night before bed. The treatment period herein is ideally of sufficient time for the skin care actives to improve the appearance of the skin. The treatment period may last for at least a week (e.g., about 1 week, about 2 weeks, about 4 weeks, about 8 weeks, or even about 12 weeks). In some instances, the treatment period will extend over multiple months (i.e., about 3-12 months). In some instances, the composition may be applied most days of the week (e.g., at least 4, 5 or 6 days a week), at least once a day or even twice a day during a treatment period of at least 2 weeks, 4 weeks, 8 weeks, or 12 weeks.

The serum composition can be applied as follows:
First, cleanse skin to remove dirt, oil, or makeup.
Then, dispense desired amount, generally 4-5 drops, of serum into palm and smooth evenly over face, neck, and/or a portion or a combination thereof.
Optionally, moisturize to protect your skin. For daytime, use a formula with SPF.

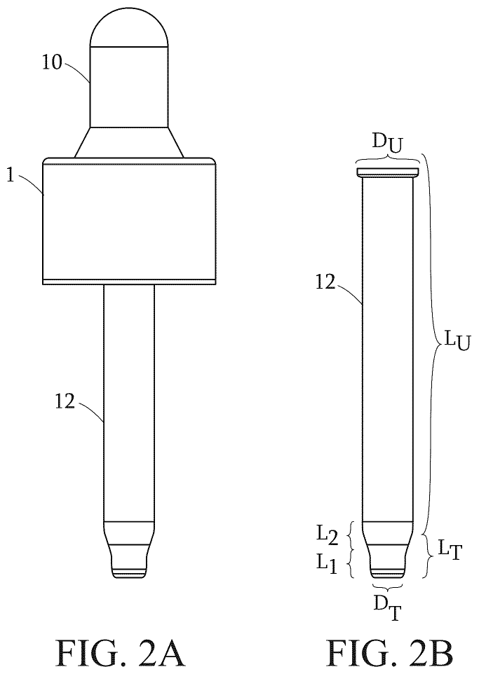

The serum composition can be stored in and dispensed from a bottle adapted for storing a serum composition with a dropper. The bottle can be any suitable material including plastic, in particular recyclable plastic material such as PET, HDPE, PP, LDPE, and/or glass. FIG. 2A is a front view of dropper 1 comprising bulb 10 and a pipette 12 coupled to the bulb. FIG. 2B is a front view of pipette 12 with a tip with length L_{T} and an internal diameter D_{T} and a tube with length L_{U} and an internal diameter D_{U}, with L_{T}=L₁+L₂/2. In a standard pipette, L_{T} can range from about 3 mm to about 12 mm, L_{U} can range from about 55 mm to about 80 mm, D_{T} can range from about 1 mm to about 2.3 mm, and D_{U} can range from about 4 mm to about 6 mm.

In other examples, the serum composition can be stored in and dispensed from a bottle with a pump.

Method of Making: Finetuned High Shear Method

A water-phase is made that includes all the water-phase ingredients except the polymer. The PemuPur™ START polymer can be added directly to the water phase without heat. Mixing speed should be watched and adjusted during the polymer dispersion/activation step to maintain proper flow without creating aeration or spills as the formula becomes thicker. The water phase can be made using a mixed flow impeller blade and a T25 Ultra-Turrax® dispersing mill simultaneously to disperse the polymer into the water phase to form the water/PemuPur™ phase. If the composition is an emulsion, the oil phase ingredients are mixed in a separate container until uniform to form the oil phase. Then the oil phase ingredients are added to the water phase/PemuPur™ phase and the dispersing mill for high shear can be used to incorporate the dispersed phase/oil phase. The result is a uniform serum where the ingredients, particularly the microcrystalline cellulose, are evenly sheared without visible particles.

It was found that to form a stable emulsion with a relatively high amount (e.g., 7-15%) of oil/silicone (e.g., 5 cst dimethicone, coconut alkanes, capric/caprylic triglyceride, coco-caprylate/caprate, isoamyl laurate, squalane, isohexadecane, and isopropyl isostearate, or combinations thereof) both a mixed flow impeller blade and a dispersing mill were needed to be used simultaneously to form a stable, uniform, serum without visible oil droplets or particles in a reasonable amount of time (e.g., approximately 5-10 minutes). It was also found that in single phase compositions, using both the impeller blade and the dispersing mill simultaneously could help form a stable, uniform, serum without visible particles in a reasonable amount of time (e.g., approximately 5-10 minutes). The combination of lower polymer levels, higher shear, and lower oil quantities led to the best quality emulsions after repeated experimentation. In some limited cases, certain oils were not as successful for emulsion stability due to their unique chemistry, such as 15% coconut alkanes which had some oil that separated. But the lower level of coconut alkanes at 7% was successfully stable.

### Definitions

As used herein, “clean” refers to cosmetic compositions that are formulated without the following ingredients: Acrylate Monomers, Aluminum Salts, Animal Musk's/Fats/Oils, Benzophenones and related compounds, Butoxyethanol, Butylated hydroxyanisole (BHA), Butylated hydroxytoluene (BHT), Carbon Black or Black 2, Coal Tar, Ethanolamines, Formaldehyde and Formaldehyde-releasing agents, Hydroquinone, Lead and Lead Acetate, Mercury and Mercury Compounds (Thimerisol), Methoxyethanol, Methyl Cellosolve, Methylchloroisothiazolinone and Methylisothiazolinone, Mineral Oil, Nanomaterials, Chemical sunscreens (including oxybenzone, avobenzone, and octinoxate), Parabens (including butyl-, methyl-, and propylparaben), Petrolatum and Paraffin, Phenoxyethanol, Phthalates, Plastic Microbeads, Polyacrylamide and Acrylamide, Resorcinol, Retinyl Palmitate, Styrene, Sulfates (including sodium lauryl sulfate and sodium laureth sulfate), Talc, Toluene, Triclosan and Triclocarban, Acetaldehyde, Acetone, Acetonitrile, Benzalkonium chloride, Bisphenol A (BPA), Ethylenediaminetetraacetic acid (EDTA), Methylene chloride, Polytetrafluoroethylene (PTFE), Perfluorooctanoic acid (PFOA), 1,4 Dioxane, Octinoxate, Nitromusks and Polycyclic Musks, PFAS compounds, Ethoxylated Ingredients, Polyethylene glycol (PEG), Cyclic Silicones, volatile linear silicones including silicone elastomers which contain volatile linear silicones, and derivatives and combinations thereof.

As used herein, “formulated without” means that the ingredient is not intentionally added. However, “formulated without” does not guarantee “100% free from” since trace contaminants are possible.

As used herein, “molecular weight” refers to weight average molecular weight unless otherwise stated. Molecular weights are measured using the industry standard method of gel permeation chromatography (“GPC”).

As used herein, “natural” refers to cosmetic ingredients obtained only from plants, animals, micro-organisms, or minerals according to International Organization for Standardization 16128-1, Part 1, [symbol] 2.1 (February 15, 2016). Ingredients obtained from fossil fuels are excluded from the definition. A non-limiting list of “natural” ingredient sources can include olive, palm, corn, palm, rapeseed, soybean, sunflower, coconut, tallow, canola, castor, and combinations hereof.

As used herein, “naturally derived” refers to cosmetic ingredients of greater than 50% natural origin by molecular weight, by renewable carbon content or by any other relevant methods, obtained through defined chemical and/or biological processes with the intention of chemical modification according to ISO 16128-1, Part 1, [symbol] 3.1 (February 15, 2016). ISO 16128-1, Appendix B includes a non-limiting list of chemical and biological processes for derived natural ingredients, which is incorporated by reference.

As used herein, “skin care” means regulating and/or improving a skin condition. Some nonlimiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin; preventing damage to skin via antioxidant approaches, including UV A and UV B induced damage, preventing formation of comedomes, balancing the skin microbiome or preventing acne.
As used herein, “skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

As used herein, “skin care composition” means a composition that includes a skin care active and regulates and/or improves skin condition.

As used herein, “substantially free” means the specific material may be present in small amounts that do not materially affect the basic and novel characteristics of the compositions according to the disclosure. For instance, there may be less than less than 2%, less than 1.5%, less than 1%, less than 0.5%, less than 0.25%, less than 0.1%, less than 0.05%, less than 0.01%, and/or none of the specified material.

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 21° C. and at ambient conditions, where “ambient conditions” means conditions under about 1 atmosphere of pressure and at about 50% relative humidity. All weights as they pertain to listed ingredients are based on the active level and do not include carriers or by-products that may be included in commercially available materials, unless otherwise specified.

All ranges and amounts given herein are intended to include sub-ranges and amounts using any disclosed point as an end point. Thus, a range from 1-5, includes specifically 1, 2, 3, 4, and 5, as well as sub ranges such as 2-5, 3-5, 2-3, 2-4, 1-4, etc. All ranges and values disclosed herein are inclusive and combinable. Further, it is expressly contemplated that any value or point reported in the Examples that falls within a range described herein can serve as a minimum or maximum value to derive a subrange.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

It is to be understood that, as used herein the terms “the,” “a,” or “an,” mean “at least one,” and should not be limited to “only one” unless explicitly indicated to the contrary. Thus, for example, reference to “a portion” includes examples having two or more such portions unless the context clearly indicates otherwise.

While various features, elements or steps of particular embodiments may be disclosed using the transitional phrase “comprising,” it is to be understood that alternative embodiments, including those that may be described using the transitional phrases “consisting of” or “consisting essentially of,” are implied. Thus, for example, implied alternative embodiments to a method that comprises A+B+C include embodiments where a method consists of A+B+C and embodiments where a method consists essentially of A+B+C. As described, the phrase “at least one of A, B, and C” is intended to include “at least one A or at least one B or at least one C,” and is also intended to include “at least one A and at least one B and at least one C.”

### Test Methods

### Color Test Method

For the purposes of the present invention, color is defined according to a value on the CIELAB color system, which is based on XYZ color system, defined by the Commission Internationale de l′Eclairage (CIE system) to provide a manner of objectively representing perceived color and color differences. X, Y, and Z can be expressed in a variety of manners, or “scales,” one of which is the Hunter Scale. The Hunter scale has three variables, L, a, and b, which correlate mathematically to X, Y, and Z, and is described by Robertson, A.R. in “The CIE 1976 Color Difference Formulas,” Color Research Applications, vol. 2, pp. 7-11 (1977).

To measure the color of the compositions of the present invention, approximately 8 mL of the product to be tested is transferred into a petri dish (35×10 mm disposable) using a syringe. The Petri dish is then placed on the black half of a standard opacity chart (Form N2A, Lencta Company of Manwah, NJ or the equivalent thereof, of which the top half is black and the bottom half is white). The color (L, a, and b values) of the product in the petri dish is then measured using an integrated sphere spectrophotometer with settings selected to exclude specular reflection. The value for “a” correlates to a value along the red-green (horizontal) axis, and the value for “b” correlates to a value along the blue-yellow (vertical) axis. For example, a yellow-colored sample will have a positive b-value, whereas a green colored sample will have a negative a-value. A more positive or negative value represents a more intense color. The value for “L” is an indicator of lightness and/or darkness, and correlates to a value along the z-axis, which is perpendicular to both the horizontal and vertical axes.

### HPLC Test Method

This method provides a suitable means for determining the amount of RP loss in a composition.

A sufficient quantity of the test composition is placed in a controlled environment chamber/room at the desired temperature, humidity, and duration. When accelerated aging is complete, the sample is removed from the controlled environment, equilibrated to room temperature (21° C.±2° C.) for 24 hours, and then measured for chemical analysis shortly after. Note that retinoids are light sensitive and should not be exposed to direct light. All test compositions are mixed thoroughly before sampling.

The amount of RP loss is determined on a % w/w basis by HPLC (isocratic elution) as follows. The HPLC column(s) is conditioned in accordance with conventional practices. Chromatographic Conditions
1. Column: C18 (5 micron), 250 mm×4.6 mm
2. Mobile Phase (Eluent): methanol/2-propanol (70/30 v/v)
3. Column temperature: approximately 25° C. (Ambient)
4. UV wavelength: 280 nm
5. Injection volume: 20 microliters
6. Flow rate: 1.0 ml/min
7. Run time: approximately 22.0 minutes
8. RP retention time: 6 minutes

### Mobile Phase Preparation

Prepare 1 L of Mobile Phase (Eluent) by combining 700 mL of methanol with 300 mL of 2-propanol.

### External Standard Preparation (prepare fresh on day of use)

In a 25 ml amber flask, dissolve 25 mg of retinyl propionate in 10 ml of Mobile Phase. Avoid exposing the RP to light. Dilute to volume with Mobile Phase. Transfer a 5.0 ml aliquot to a 50 ml amber flask and dilute to volume with mobile phase. Mix well. Filter about 1 ml of the external standard thru a suitable 0.45 micron filter (e.g., Whatman GD/X) into an amber HPLC sample vial.

### HPLC Sample Preparation

Using a 1-cc tuberculin syringe, weigh and transfer 500 mg of the sample composition into a 25 ml amber volumetric flask. Add 10 mL of Mobile Phase and vortex on high for 2 minutes or until product is completely dispersed. Dilute to volume with mobile phase and mix well. Filter approximately 1 ml into an auto-sampler vial using a syringe filter (e.g., Whatman GD/X filter unit). Perform 20 injections using the condition described.

(A)/(B)×(B)/(W)×(DF)×100=retinyl propionate, % w/w
Where,
A=peak area of retinyl propionate for the sample
B=peak area of retinyl propionate for the calibration
C=retinyl propionate standard weight in mg
W=sample weight, mg
DF=Dilution Factor (e.g., 0.1)

### Rheology Test Method

This method provides a suitable means of measuring the viscosity of the compositions herein. The instrument used in this method is a TA Instruments brand rheometer, or equivalent. The instrument is set up to conduct a rotational ramp under controlled stress conditions, from 0.01 to 3000 Pa over 240 seconds, using a flat steel bottom plate for the lower geometry and a 1.008 degree steel cone plate for the upper geometry with a 40 mm diameter, and a 26 micron gap. The temperature is set to 25° C. The instrument protocol is set up to collect 100 data points per decade in a logarithmic distribution, with a setting to stop the measure when shear rates exceed 110 s⁻¹.

After conducting an automated gap calibration, approximately 0.5 mL of a sample is placed on the center of the plate. The plate is positioned to provide a gap of 126 microns, and any excess material squeezed out of the perimeter gap of the parallel plates is carefully trimmed away. The plate is then moved the final 100 microns to provide a 26 micron gap, and the measurement is initiated. At the end of the run, the data file is saved for subsequent plotting and analysis. The viscosity is reported in units of Pascal-Seconds (Pa·s) as the value at or near a given shear rate.

### Tack Method

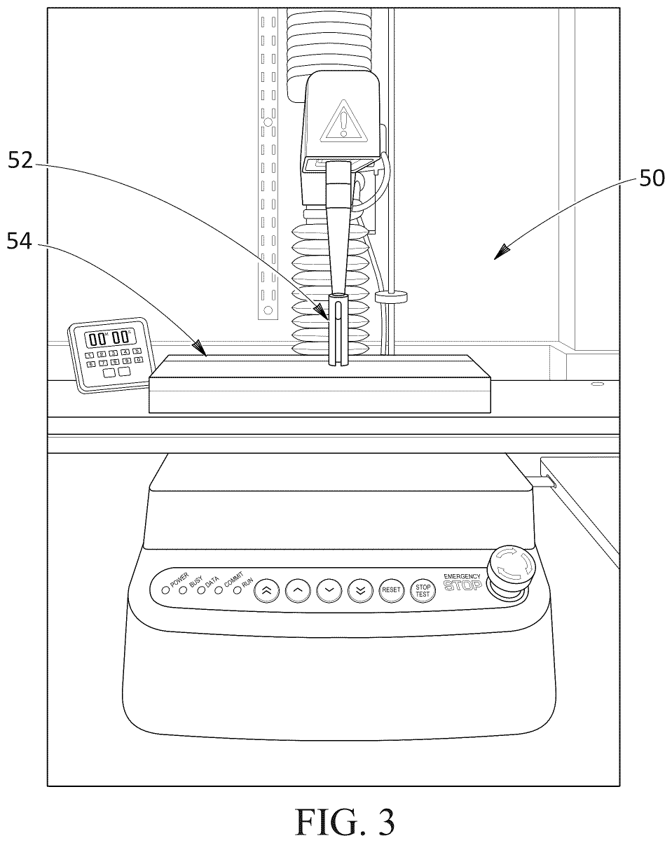

This method provides a suitable means for determining the tackiness of the compositions described herein. The method uses a texture analyzer to contact a probe with a film formed from the composition. The texture analyzer then measures the force needed to separate the probe from the composition film. Tack Force and Time Weighted Force Area can be determined by this method. The Tack Method is configured to run for a period of 100 minutes. It is believed, without being limited by theory, that the tackiness properties exhibited by a composition over the course of the Tack Test approximate the tackiness properties exhibited by the composition during the first few minutes (e.g., less than 10 minutes, between 30 seconds and 5 minutes, or between 1 minute and 3 minutes) of use of the composition by a user. It is also believed, without being limited by theory, that having a suitable tack force at the later time periods in the test (e.g., 60 minutes, 80 minutes, and/or 100 minutes), influences the moisturization perception of the user more than the tack force at earlier time periods in the test. Thus, time weighted force area is used to weigh the tack force measurements toward the later time points, and thereby provide a more accurate prediction of the moisturization signal perceived by a user. FIG. 3 illustrates an exemplary test setup for the Tack Test. A TA.XT2i brand texture analyzer (available from Texture Technologies Corporation, MA) 50 or equivalent is used to measure the tack of a film formed from a composition of interest. The texture analyzer is equipped with a tack probe 52 in the form of a 12.75 mm diameter acrylic cylinder with a flat surface. During testing, the flat surface of the probe 52 is contacted with the surface of a film of the composition. Thus, the flat surface of the probe and the surface of the film must be parallel to one another during testing to ensure there is sufficient contact by the film across the effective test surface of the probe 52. The film is made by filling or overfilling (followed by draw down) a rectangular channel 54 (e.g., 25 cm long×30 mm wide a 0.25 mm deep) with the composition to be tested.

The test is conducted using an adhesive test protocol with a pretest speed of 0.10 mm/second, a test speed of 0.10 mm/sec and a post-test speed of 1.0 mm/sec. The applied force is 200 g, the return distance is 4 mm and the contact time is 5.0 sec. The trigger type to designate sample contact is set to automatic and the trigger force is 5.0 g. The test is run and at the following time increments immediately after the film is prepared: <1 minute (i.e., immediately following preparation of the film), 10, 20, 30, 40, 50, 60, 80, and 100 minutes. Each time point is run on a previously undisturbed/untested area of the sample. Each sample is run in triplicate and the averages are recorded.

Data extraction uses the portion of data that is collected as the probe pulls upward out of the sample. The tack force is the peak force of each test run. Time weighted force area is determined by the following formula:

$$tiime weighted force area = ( t 1 - t 2 ) * [ \frac{P 1 - P 2}{200} ] * [ ( t 1 + t 2 ) / 2 ]$$

Where:
t1=the later of the two times in the time range at which the measurement was taken;
t2=the earlier of the two times in the time range at which the measurement was taken;
P1=peak force in grams at time 1 in the pair of times being calculated; and
P2=peak force in grams at time 2 in the pair of times being calculated. When calculating the difference between t1 and t2 or P1 and P2, the absolute value of the result is used to calculate time weighted force area.

Time weighted force area is reported as the sum of the individual time weighted force area values calculated for each of the 8 time intervals (<1 min to 10 min, 10 min to 20 min, 20 min to 30 min, etc.).

### EXAMPLES

The following data and examples are provided to help illustrate the skincare compositions described herein. The exemplified compositions are given solely for the purpose of illustration and are not to be construed as limitations of the present disclosure, as many variations thereof are possible without departing from the spirit and scope of the disclosure. All parts, percentages, and ratios herein are by weight unless otherwise specified.

In Table 1, Table 2, and Table 4, the time weighted force area is calculated according to the Tack Test Method, described herein.

The Examples in Table 1, below, were prepared by conventional methods of making topical skin care water-phase serums. Example A and Example B were made by combining all ingredients except the polymers and glycerin (e.g., everything except sodium hyaluronate, glycerin, carrageenan, Tremella fuciformis polysaccharide, and xanthan gum) in a container and mixed until uniform to create a water phase. These remaining ingredients were combined in a separate container and mixed until uniform to form a glycerin/polymer phase. The glycerin/polymer phase was then slowly added to the water phase and mixed until uniform, resulting in the water-phase serum.

Example C was made by first combining the water and hydroxyethylcellulose in a container and mixing until uniform. Then the NaOH was added to the container, followed by sodium lactate and lactic acid, then the niacinamide, histidine, trehalose, and lysine were added. Finally, in a separate container the glycerin and the remaining ingredients were combined and mixed until uniform to form the glycerin/polymer phase. The glycerin/polymer phase was then slowly added to the water phase and mixed until uniform, resulting in the water-phase serum.

TABLE 1

| | Ex. A | Ex. B | Ex. C |
|---|---|---|---|
| | (Sodium | (Xanthan | (Hydroxyethyl- |
| | Hyaluronate | Gum | cellulose |
| | Water-phase | Water-phase | Water-phase |
| | Serum) | Serum) | Serum) |
| Water | Q.S. | Q.S. | Q.S. |
| Glycerin (99.7% glycerin | 6 | 4 | 5 |
| solution) | | | |
| Niacinamide | 2 | 2 | 2 |
| Sodium Lactate¹ | 1.3 | 1.3 | 1.3 |
| L-Lactic Acid² | 1.8 | 1.8 | 1.8 |
| Dihydrate Trehalose³ | 1 | 1 | 1 |
| Panthenol⁴ | 1 | 1 | 1 |
| Xanthan Gum | | 0.7 | |
| Sodium Hyaluronate⁵ | 0.8 | 0.2 | 0.2 |
| Hydroxyethylcellulose⁶ | | | 0.8 |
| Tremella fuciformis | 0.1 | 0.1 | 0.1 |
| Polysaccharide⁷ | | | |
| Carrageenan⁸ | 0.3 | 0.1 | 0.1 |
| Microcrystalline Cellulose | | | |
| (and) Sphingomonas | | | |
| Ferment Extract (and) | | | |
| Cellulose Gum⁹ | | | |
| Retinyl Propionate²¹ | | | |
| Carnosine¹⁰ | | | |
| Sodium Sulfite | | | |
| Coco-caprylate/caprate¹¹ | | | |
| Dimethicone 5 cst¹² | | | |
| Ethyl Ascorbic Acid¹³ | | | |
| Cocos nucifera fruit | | | |
| juice¹⁴ | | | |
| Camellia sinensis Leaf | 0.1 | 0.1 | 0.1 |
| Extract¹⁵ | | | |
| Lycine HCl¹⁶ | 0.1 | 0.1 | 0.1 |
| Histidine¹⁷ | 0.05 | 0.05 | 0.05 |
| Phenoxyethanol (99% | | | |
| phenoxyethanol | | | |
| solution)¹⁸ | | | |
| Benzyl Alcohol (95-100% | | | |
| benzyl alcohol solution) | | | |
| Sodium Hydroxide (54% | | | 0.02 |
| NaOH Solution) | | | |
| Ethylhexylglycerin¹⁹ | | | |
| Hydroxyacetophenone²⁰ | 0.15 | 0.15 | 0.15 |
| Time Weighted Force | 32,193 | 23,342 | 23,585 |
| Area (g) | | | |

TABLE 2
Control Serum

| | |
|---|---|
| Product | Native Moisturizing Facial Serum |
| | Lot # 2019A1042A |
| INCI | water, glycerin, niacinamide, panthenol, cocos nucifera |
| | (coconut), fruit juice, sodium hyaluronate, 3-o-ethyl |
| | ascorbic acid, xanthan gum, benzyl alcohol |
| Time Weighted | 30,667 |
| Force Area (g) | |

The serum compositions in Table 1, were tested for six-weeks on 14 consumers, both men and women, ages 18-56, in Cincinnati, Ohio, USA. Each consumer tested each product, one at a time, for one-week. Immediately after using each product, the consumers provided qualitative data in a focus group. The study ran for four consecutive weeks. The results from this study are summarized in Table, below.

TABLE 3

| Control Serum | Ex. A | Ex. B | Ex. C |
|---|---|---|---|
| Key Conclusions | Key Conclusions | Key Conclusions | Key Conclusions |
| The Control Serum | Although having a | The reviews were | It was clear that |
| did not deliver | thicker viscosity, | mainly focused on | consumers preferred |
| consumer delight. | Example A was | delayed absorption, | the |
| The panelists thought | perceived similarly to | excessive shine, | hydoxyethylcellulose |
| the product was too | the Control Serum. | peeling of the serum, | variant over the other |
| thin and the excessive | Stickiness seemed to | and dry skin, and | serum formulations in |
| stickiness left the | be a turn-off, | there were also | this test. |
| face and hand feeling | followed by a lack of | questions about | One pump was more |
| unclean. | hydration. | bubbles in the | than enough for the |
| | The thin viscosity | formula. | face and neck. |
| | drove the consumers | Many desired | |
| | to use more product | attributes like | |
| | to feel the application | hydration, dewy and | |
| | was sufficient. | glowing were absent | |
| | | during use. | |
| | | Sticky/tacky seems to | |
| | | increase with other | |
| | | skincare products | |
| | | (i.e., moisturizer). | |

The consumer test indicated that Example C, which included a hydroxyethylcellulose polymer, was preferred over the other serums tested. Example C had a time weighted force area greater than 2.3×10⁴ g, which indicated that the serum would feel too heavy and/or sticky to be consumer preferred. Therefore, it was determined that another polymer system needed to be identified that would further reduce drying time, increase absorption, maintain hydration, and further reduce stickiness to formulate consumer preferred serum. Since hydroxyethylcellulose was the most successful polymer in this consumer test, additional cellulose-based polymers were tested. Table 4, includes Examples D, E, and F. These examples contain microcrystalline cellulose,

Sphingomonas ferment extract, and cellulose gum. It was found that these formulas had a significant reduction in time weighted force area, as compared to the Control Serum and Examples A, B, and C.

The examples in Table 4 to Table 6 and Table 9, below, were prepared using the Finetuned High Shear Method, described herein. The following examples were made by combining the oil phase ingredients in a small separate container and mixing until uniform. In a separate main mix container, all remaining ingredients, except the PemuPur™ START Polymer, were added to water and mixed until uniform to form a water phase. Once the water phase was homogeneous, the PemuPur™ START Polymer was added to the main mix container. The water phase/PemuPur™ mixture was mixed/milled with an overhead mixed flow impeller blade (available from INDCO) and a T25 Ultra-Turrax handheld mill until homogeneous to form the water phase/PemuPur™ phase. Then, the oil phase was added to the water phase/PemuPur™ and was further mixed/milled with the overhead mixed flow impeller blade and the handheld mill to form the emulsion serum. The RPM on the overhead mixer was adjusted throughout to form a homogenous composition, but not so high that it incorporates air or sputters.

TABLE 4

| Ingredient Name | Ex. D | Ex. E | Ex. F |
|---|---|---|---|
| Water Phase: | | | |
| Water | Q.S. | Q.S. | Q.S. |
| Glycerin (99.7% glycerin | 3 | 5 | 3 |
| solution) | | | |
| Niacinamide | 2 | | 2 |
| Sodium Lactate¹ | | | |
| L-Lactic Acid² | | | |
| Dihydrate Trehalose³ | | | |
| Panthenol⁴ | 0.5 | | 0.5 |
| Xanthan Gum | | | |
| Sodium Hyaluronate⁵ | | | |
| Hydroxyethylcellulose⁶ | | | |
| Tremella Fuciformis | | | |
| Polysaccharide⁷ | | | |
| Carrageenan⁸ | | | |
| Microcrystalline Cellulose | 0.7 | 0.7 | 0.7 |
| (and) Sphingomonas | | | |
| Ferment Extract (and) | | | |
| Cellulose Gum⁹ | | | |
| Carnosine¹⁰ | | 0.2 | |
| Sodium Sulfite | | 0.1 | |
| Ethyl Ascorbic Acid¹³ | | | |
| Cocos nucifera fruit | | | |
| juice¹⁴ | | | |
| Camellia sinensis Leaf | | | |
| Extract¹⁵ | | | |
| Lycine HCl¹⁶ | | | |
| Histidine¹⁷ | | | |
| Phenoxyethanol (99% | 0.375 | 0.375 | 0.375 |
| phenoxyethanol | | | |
| solution)¹⁸ | | | |
| Benzyl Alcohol (95-100% | | | |
| benzyl alcohol solution) | | | |
| Sodium Hydroxide (54% | | | |
| NaOH Solution) | | | |
| Ethylhexylglycerin¹⁹ | 0.2 | 0.2 | 0.2 |
| Hydroxyacetophenone²⁰ | 0.15 | 0.15 | 0.15 |
| Disodium EDTA | 0.05 | 0.05 | 0.05 |
| Oil Phase: | | | |
| Retinyl Propionate²¹ | | 0.3 | |
| Coco-caprylate/caprate¹¹ | 9 | 9 | |
| Dimethicone 5 cst¹² | | | 9 |
| Time Weighted Force | 16,581 | 15,793 | 13,965 |
| Area (g) | | | |

It was found that the time weighted force area for Examples D, E, and F, which contained a polymer system comprising microcrystalline cellulose, Sphingomonas ferment extract, and cellulose gum were significantly lower than the Control and Examples A, B, and C. This indicated that Examples D, E, and F are less tacky and may be consumer preferred.

Ex. G-I, described in Table 5 and Table 6, below, are examples of serums containing a polymer system that includes microcrystalline cellulose, Sphingomonas ferment extract, and cellulose gum. These examples were selected from a plurality of examples and represent a range of rheology profiles that are consumer acceptable. Example G is a water-phase composition that had the lowest viscosity across all shear viscosities, as of the date of the provisional filing. Example H is the emulsion that had the lowest viscosity across all shear rates of any of the emulsions tested, as of the date of the provisional filing. Example I is the emulsion that had the highest viscosity across all shear rates of any of the emulsions tested, as of the date of the provisional filing. There can be consumer preferred physically stable formulas with higher and lower viscosities than those exemplified in Examples G, H, and I. Example D in Table 4, is a formula that is has a median amount of oil and PemuPur™ START polymer and can be interesting to compare to Examples G, H, and I.

TABLE 5
Single Phase Water-Serum containing Microcrystalline Cellulose,
Sphingomonas Ferment Extract, and Cellulose Gum

| | Ex. G |
|---|---|
| Water | Q.S. |
| Glycerin (99.7% glycerin) | 5 |
| Niacinamide | |
| Sodium Lactate¹ | |
| L-Lactic Acid² | |
| Dihydrate Trehalose³ | |
| Panthenol⁴ | |
| Xanthan Gum | |
| Sodium Hyaluronate⁵ | |
| Hydroxyethylcellulose⁶ | |
| Tremella Fuciformis Polysaccharide⁷ | |
| Carrageenan⁸ | |
| Microcrystalline Cellulose (and) Sphingomonas | 0.5 |
| Ferment Extract (and) Cellulose Gum⁹ | |
| Retinol Propionate | |
| Carnosine¹⁰ | |
| Sodium Sulfite | |
| Coco-caprylate/caprate¹¹ | |
| Capric/caprylic triglyceride²² | |
| Dimethicone 5 cst¹² | |
| Ethyl Ascorbic Acid¹³ | |
| Cocos nucifera fruit juice¹⁴ | |
| Camellia sinensis Leaf Extract¹⁵ | |
| Lycine HCl¹⁶ | |
| Histidine¹⁷ | |
| Phenoxyethanol (99%)¹⁸ | 0.375 |
| Benzyl Alcohol (95-100%) | |
| Sodium Hydroxide (54%) | |
| Ethylhexylglycerin¹⁹ | 0.2 |
| Hydroxyacetophenone²⁰ | 0.15 |
| Disodium EDTA | 0.05 |

TABLE 6
Emulsion Serums containing Microcrystalline Cellulose,
Sphingomonas Ferment Extract, and Cellulose Gum

| | Ex. H | Ex. I |
|---|---|---|
| Water Phase: | | |
| Water | Q.S. | Q.S. |
| Glycerin (99.7% glycerin) | 5 | 5 |
| Niacinamide | | 10 |
| Sodium Lactate¹ | | |
| L-Lactic Acid² | | |
| Dihydrate Trehalose³ | | |
| Panthenol⁴ | | |
| Xanthan Gum | | |
| Sodium Hyaluronate⁵ | | |
| Hydroxyethylcellulose⁶ | | |
| Tremella Fuciformis Polysaccharide⁷ | | |
| Carrageenan⁸ | | |
| Microcrystalline Cellulose (and) Sphingomonas | 0.5 | 0.7 |
| Ferment Extract (and) Cellulose Gum⁹ | | |
| Retinol Propionate | | |
| Carnosine¹⁰ | | |
| Sodium Sulfite | | |
| Ethyl Ascorbic Acid¹³ | | |
| Cocos nucifera fruit juice¹⁴ | | |
| Camellia sinensis Leaf Extract¹⁵ | | |
| Lycine HCl¹⁶ | | |
| Histidine¹⁷ | | |
| Phenoxyethanol (99%)¹⁸ | 0.375 | 0.375 |
| Benzyl Alcohol (95-100%) | | |
| Sodium Hydroxide (54%) | | |
| Ethylhexylglycerin¹⁹ | 0.2 | 0.2 |
| Hydroxyacetophenone²⁰ | 0.15 | 0.15 |
| Disodium EDTA | 0.05 | 0.05 |
| Oil Phase: | | |
| Retinyl Propionate²¹ | | |
| Coco-caprylate/caprate¹¹ | | |
| Dimethicone 5 cst¹² | | |
| Capric/caprylic triglyceride²² | 15 | 15 |

TABLE 7
Commercially Available Serum Compositions

| Product | INCI |
|---|---|
| Olay ® ProX | water, niacinamide, polymethylsilsesquioxane, dimethicone, |
| Lot #91780386MA | glycerin, butylene glycol, methyl gluceth-20, panthenol, |
| | polysorbate 20, undecylenoyl phenylalanine, aminomethyl |
| | propanol, acrylates/vinyl isodecanoate crosspolymer, benzyl |
| | alcohol, dipotassium glycyrrhizate, allantoin, laureth-4, |
| | methylparaben, dimethiconol, silica dimethyl silylate, |
| | sodium benzoate, disodium edta, citric acid, laminaria |
| | saccharina extract, glyceryl acrylate/acrylic acid copolymer, |
| | propylene glycol, pvm/ma copolymer |
| Olay ® Vitamin C + Peptide 24 | water, glycerin, dimethicone, niacinamide, lactic acid, |
| Brightening Serum (Olay | polyacrylate crosspolymer-6, 3-o-ethyl ascorbic acid, |
| VitC) | palmitoyl pentapeptide-4, panthenol, trehalose, PEG-11 |
| Lot # 2085TN60 | methyl ether dimethicone, sodium lactate, sodium benzoate, |
| | fragrance |
| Olay ® Regenerist ® MAX | water, glycerin, dimethicone, niacinamide, butylene glycol, |
| Wrinkle Serum with Peptides | palmitoyl pentapeptide-4, palmitoyl dipeptide-7, tropaeolum |
| (Olay Wrinkle MAX) | majus flower/leaf/stem extract, panthenol, tocopheryl |
| Lot # 2078TN30 | acetate, hydroxyacetophenone, dimethiconol, polysorbate 20, |
| | disodium edta, dimethicone crosspolymer, carbomer, |
| | acrylates/c10-30 alkyl acrylate crosspolymer, aminomethyl |
| | propanol, phenoxyethanol |
| Olay ® Regenerist ® Retinol 24 | water, dimethicone, glycerin, retinol, retinyl propionate, |
| Night Serum (Olay Ret24) | niacinamide, palmitoyl pentapeptide-4, dimethiconol, c13-14 |
| Lot # 2038TN35 | isoparaffin, laureth-4, polysorbate 20, laureth-7, disodium |
| | edta, dimethicone crosspolymer, polyacrylamide, titanium |
| | dioxide, mica, dmdm hydantoin, iodopropynyl |
| | butylcarbamate |
| Olay ® Collagen Peptide 24 | water, dimethicone, niacinamide, glycerin, panthenol, |
| (Olay Collagen) | palmitoyl pentapeptide-4, tremella fuciformis sporocarp |
| Lot # 2100TN33 | extract, hydroxyacetophenone, c13-14 isoparaffin, laureth-4, |
| | polysorbate 20, laureth-7, disodium EDTA, polyacrylamide, |
| | dimethicone crosspolymer, phenoxyethanol |
| Olay ® Hyaluronic + Peptide | water, glycerin, dimethicone, niacinamide, sodium |
| 24 Serum (Olay ® HA) | hyluronate, palmitoyl pentapeptide-4, panthenol, |
| Lot # 2013TN32 | ethylhexlglycerin, hydroxyacetophenone, c13-14 isoparaffin, |
| | dimethiconol, polysorbate 20, laureth-4, laureth-7, disodium |
| | edta, polyacrylamide, phenoxyethanol |

The Rheology Test Method was performed for the compositions in Table 1, Example D in Table 4, and the compositions in Table 5, Table 6, and Table 7 to produce individual rheology curves. Each curve was put on the same graph with logarithmic x and y axes to compare the curves for each serum composition, as shown in FIG. 1. FIG. 1 shows that the examples with a polymer system containing microcrystalline cellulose, Sphingomonas ferment extract, and cellulose gum (“inventive examples”) had a rheology profile that was about the same or better than current commercially available products.

It was determined that the ideal rheology curve for a serum product meets the following rheology criteria:
At 0.01 s⁻¹ shear rate, the viscosity can be less than or equal to 300 Pa·s
At 20 s⁻¹ shear rate the viscosity can be less than or equal to 0.6 Pa·s
Between 0.01 s⁻¹ and 0.1 s⁻¹ shear rate, the slope (with viscosity on the y-axis) is greater than −600 and less than 0.

Table 8, below, summarizes the results. The viscosity at 20 s⁻¹ and viscosity at 0.01 s⁻¹ were determined using the Rheology Method, described herein and the slope was calculated using the equation, described herein.

TABLE 8

| | Viscosity at | | Viscosity at | | Slope between | |
|---|---|---|---|---|---|---|
| | 20 s⁻¹ (Pa · s) | | 0.01 s⁻¹ (Pa · s) | | 0.01 s⁻¹ and 0.1 s⁻¹ | |
| | Measured | Meets | Measured | Meets | Calculated | Meets |
| | Value | Criteria? | Value | Criteria? | Value | Criteria? |
| Olay ® ProX | 0.65 | No | 142.95 | Yes | −1163.97 | No |
| Olay VitC | 0.91 | No | 374.09 | No, too high | −2704.13 | No |
| Olay Wrinkle | 0.62 | No | 70.53 | Yes | −591.60 | Yes |
| MAX | | | | | | |
| Olay Ret24 | 1.27 | No | 177.95 | Yes | −1591.26 | No |
| Olay Collagen | 1.41 | No | 151.95 | Yes | −1279.61 | No |
| Olay HA | 5.55 | No | 48.33 | Yes | −181.30 | Yes |
| Control Serum | 0.27 | Yes | 31.93 | Yes | −154.37 | Yes |
| Ex. A | 1.021 | No | 4.416 | Yes | −9.32 | Yes |
| Ex. B | 1.14 | No | 125.56 | Yes | −644.31 | No |
| Ex. C | 2.45 | No | 29.84 | Yes | −27.58 | Yes |
| Ex. D | 0.45 | Yes | 85.78 | Yes | −472.07 | Yes |
| Ex. G | 0.21 | Yes | 29.53 | Yes | −167.83 | Yes |
| Ex. H | 0.59 | Yes | 116.73 | Yes | −598.16 | Yes |
| Ex. I | 0.31 | Yes | 46.39 | Yes | −234.35 | Yes |

The Control Serum and Examples D and G-I met the rheology criteria. However, the Control is not consumer preferred because consumers found the composition to be too tacky (see Table 1 and accompanying text). It is believed that Examples D and G-I will have significantly lower tack scores, as compared to the Control Serum and will be consumer preferred.

The Examples in Table 9, below, all include retinyl propionate, a retinol derivative, that is known to be unstable in skin care compositions.

TABLE 9

| | Ex. 1 | Ex. 2 | Ex. 3 | Ex. 4 | Ex. 5 | Ex. 6 |
|---|---|---|---|---|---|---|
| | (wt. %) | (wt. %) | (wt. %) | (wt. %) | (wt. %) | (wt. %) |
| Water Phase: | | | | | | |
| Water | Q.S. | Q.S. | Q.S. | Q.S. | Q.S. | Q.S. |
| Glycerin (99.7% glycerin) | 5 | 5 | 5 | 5 | 5 | 5 |
| Microcrystalline Cellulose | 0.7 | 0.7 | 0.7 | 0.7 | 0.7 | 0.7 |
| (and) Sphingomonas | | | | | | |
| Ferment Extract (and) | | | | | | |
| Cellulose Gum⁹ | | | | | | |
| Carnosine¹⁰ | 0.2 | 0.2 | 0.2 | 0.2 | 0.2 | 0.2 |
| Sodium Sulfite | 0.1 | 0.1 | 0.1 | 0.1 | 0.1 | 0.1 |
| Phenoxyethanol (99%)¹⁸ | 0.375 | 0.375 | 0.375 | 0.375 | 0.375 | 0.375 |
| Ethylhexylglycerin¹⁹ | 0.2 | 0.2 | 0.2 | 0.2 | 0.2 | 0.2 |
| Hydroxyacetophenone²⁰ | 0.15 | 0.15 | 0.15 | 0.15 | 0.15 | 0.15 |
| Disodium EDTA | 0.05 | 0.05 | 0.05 | 0.05 | 0.05 | 0.05 |
| Oil Phase: | | | | | | |
| Retinyl Propionate²¹ | 0.3 | 0.3 | 0.3 | 0.3 | 0.3 | 0.3 |
| Dimethicone 5 cst¹² | 9 | | | | | |
| Isohexadecane²³ | | 6 | | | 9 | |
| Isopropyl Isostearate²⁴ | | 3 | | | | 9 |
| Coco-caprylate/caprate²⁵ | | | 9 | | | |
| Capric/caprylic | | | | 9 | | |
| Triglyceride²² | | | | | | |

Table 10, below, shows the viscosity at 0.1^{s-1} (according to the Rheology Method, described herein), the RP % (according to the HPLC Method, described herein), and the LAB Color b-value (according to the Color Method, described herein) of Examples 1-6 in Table 9. Table 10 shows both the initial value, which was determined soon after the example was made, and a value after 2 weeks at 60° C. accelerated stability conditions.

TABLE 10

| | Viscosity at | | | | | | | | |
|---|---|---|---|---|---|---|---|---|---|
| | 0.1^{s-1} (Pa · s) | | | RP % | | | LAB Color b-value | | |
| | | 2 | | | 2 | % of | | 2 | |
| | | weeks | % | | weeks | Initial RP | | weeks | Δb- |
| | Initial | 60° C. | Change | Initial | 60° C. | Remaining | Initial | 60° C. | value |
| Ex. 1 | 42.84 | 41.17 | −3.90% | 0.301 | 0.275 | 91.4% | −4.06 | −2.43 | 1.63 |
| Ex. 2 | 41.85 | 43.42 | +3.75% | 0.315 | 0.288 | 91.4% | 0.58 | 2.26 | 1.68 |
| Ex. 3 | 45.16 | 45.51 | +7.75% | 0.278 | 0.274 | 98.6% | −1.63 | 1.07 | 2.70 |
| Ex. 4 | 44.28 | 46.83 | +5.76% | 0.333 | 0.300 | 90.1% | 2.28 | 8.12 | 5.84 |
| Ex. 5 | 43.29 | 43.17 | −0.28% | 0.307 | 0.290 | 94.5% | 1.07 | 2.35 | 1.28 |
| Ex. 6 | 41.86 | 43.30 | +3.44% | 0.341 | 0.313 | 91.8% | 1.73 | 3.50 | 1.77 |

Examples 1-6 all had greater than 90% RP remaining after the accelerated stability conditions, which is acceptable. It was surprising that the RP was so stable in compositions with PemuPur™, since this is a notoriously reactive skin care active. Furthermore, the change in viscosity and color following accelerated stability conditions was also acceptable.

Example 3, which included coco-caprylate/caprate, had the best RP stability and therefore coco-caprylate/captrate may be a preferred oil. Example 4, which included capric/caprylic triglyceride, had lower RP stability and the most significant color change and may be a less preferred oil.

Table 11, below, shows Examples 7 and 8. Example 8 includes an emollient, while Example 7 does not. Table 11 also shows the viscosity, the RP % (according to the HPLC Method, described herein), the pH, the LAB Color b-value (according to the Color Method, described herein), and the RP % (according to the HPLC Method, described herein) of Examples 7 and 8 at accelerated time/temperature conditions.

TABLE 11

| | Ex. 7 | Ex. 8 |
|---|---|---|
| | (wt. %) | (wt. %) |
| Water | Q.S. | Q.S. |
| Niacinamide | 0.005 | 0.005 |
| Disodium EDTA | 0.05 | 0.05 |
| Hydroxyacetophenone²⁶ | 0.15 | 0.15 |
| Carnosine | 0.1 | 0.1 |
| Sodium Sulfite | 0.05 | 0.05 |
| Sodium Hyaluronate²⁷ | 0.05 | 0.05 |
| Palmitoyl Pentapeptide-4 and Water | 0.125 | 0.125 |
| 99% Glycerin plant derived | 3 | 3 |
| Phenoxyethanol | 0.05 | 0.05 |
| Microcrystalline Cellulose (and) | 0.75 | 0.75 |
| Sphingomonas Ferment Extract (and) | | |
| Cellulose Gum⁹ | | |
| Dimethicone 5 cst | 14 | 14 |
| Dimethicone and Dimethicone Cross | 4 | 4 |
| Polymer²⁸ | | |
| Dimethicone 50 cst | 3 | 3 |
| Dimethicone and Dimethiconol | 1 | 1 |
| Laureth-4 | 0.2 | 0.2 |
| Polysorbate 20 | 0.2 | 0.2 |
| Retinyl Propionate | 0.325 | 0.325 |
| Dipentaerythrityl Hexa C5-9 Acid Esters²⁹ | | 8 |
| Viscosity (cP) | | |
| Initial | 5933 | 7200 |
| 2 weeks at 60° C. | 7600 | 10666 |
| 1 month at 40° C. | 5200 | 8400 |
| pH | | |
| Initial | 7.27 | 7.3 |
| 2 weeks at 60° C. | 6.93 | 6.91 |
| 1 month at 40° C. | 6.91 | 8.94 |
| b-value | | |
| Initial | 1.7 | 2 |
| 2 weeks at 60° C. | 4.74 | 5.78 |
| 1 month at 40° C. | 2.43 | 4.73 |
| % Retinyl Propionate Remaining (2 weeks at | 94.15 | 96 |
| 60° C.) | | |

The data in Table 11 suggests that the addition of the emollient increases viscosity slightly and results in a directional increase in b-value. However, both samples are likely consumer acceptable in terms of viscosity and color. Notably, both Examples 7 and 8 preserve retinyl propionate well, with 94% and 96% remaining after 2 weeks in 60° C.

Examples 7 and 8 were further tested to determine whether the emollient improved how the product feels on the skin. Tack data showed an increase in both break time and time weighted force area for Example 8, which may indicate increased moisture perception and minimized greasy/oily feeling. This technical data implies that certain consumers may prefer Example 8 due to its ability to provide a smoother and more moisturized finish. However, some consumers may prefer Example 7 becomes it may offer a lighter and fresher feel.

Suppliers and/or Ingredient Details for Table 1, Table 4 to Table 6, Table 9, and Table 11:
1. Purasal® S/HQ 60, from Corbion®
2. Purac® HiPure 90, from Corbion®
3. Trehalose 100, from Hiyashibara
4. D-panthenol (Dexpanthenol)
5. Hybloom™ (HA-T), from Bloomage Biotech
6. Keltrol® 1000, from CP Kelco®
7. Tremella Hyaluronic Acid (WSK), from Shanghai Huiwen
8. Viscarin® PC 209, from Dupont®
9. PemuPur™ START Polymer, from Lubrizol®
10. Dragosine®, from Symrise®
11. Cetiol® LC, from BASF®
12. From Dow®
13. 3-O Ethyl Ascorbic Acid, from Kobo®
14. Extrapone® Coconut Water GW, from Symrise®
15. NATPURE® XTRA GREEN TEA OP30, from Sensient®
16. L-Lysine Monohydrochloride
17. L-Histidine
18. 2-Phenoxyethanol
19. Sensiva™ SC 50, from Ashland®
20. 4′-Hydroxyacetophenone
21. Vitamin-A-Propionate 2.5 MIO IU/g in oil form that is BHT stabilized, from BASF® Human Nutrition
22. Myritol 318, from BASF®
23. Permethyl® 101A, from Presperse®
24. Schercemol™ 318 ester, from Lubrizol®
25. Cetiol® C 5C, from BASF®
26. Symsave® H, from Symrise®
27. Hybloom™, from Bloomage® Biotechnology Corp., Ltd.
28. Dowsil™ 9041, from Dow®
29. LexFeel® 350, from Inolex®

### Combinations

A. A physically stable, uniform, skin care serum composition, wherein the composition is an emulsion comprising:
   a. a continuous phase comprising a polymer system comprising microcrystalline cellulose, Sphingomonas ferment extract, and cellulose gum; a liquid carrier; and a skin care active;
   b. a dispersed phase comprising an emollient.
B. A physically stable, uniform, skin care serum composition, wherein the composition is a single phase composition comprising:
   a. a polymer system comprising microcrystalline cellulose, Sphingomonas ferment extract, and cellulose gum;
   b. a liquid carrier comprising water; and
   c. a skin care active.
C. The composition according to Paragraphs A-B, wherein the skin care active is chosen from vitamin E, vitamin C, retinol, undecylenoyl phenylalanine, niacinamide, botanicals, pre-, pro- or post-biotics, and/or derivatives and mixtures thereof.
D. The composition according to Paragraph C, wherein the skin care active comprises a retinol and/or derivatives thereof.
E. The according to Paragraph D, wherein the composition comprises greater than or equal to 75%, preferably greater than or equal to 80%, more preferably greater than or equal to 85%, and even more preferably greater than or equal to 90% of the retinyl propionate remaining as compared to the amount of retinyl propionate at the time of manufacture according to the HPLC Method.
F. The composition according to Paragraphs A-E, comprising:
   a. a viscosity of less than or equal to 300 Pa·s at 0.01 s⁻¹ according to the Rheology Test Method; and
   b. a viscosity of less than or equal to 0.6 Pa·s at 20 s⁻¹ according to the Rheology Test Method.
G. The composition according to Paragraphs A-F, wherein the composition comprises a slope between 0.01 s⁻¹ and 0.1 s⁻¹ shear rate greater than −600 and less than 0.
H. The composition according to Paragraphs A-G, comprising a viscosity at 0.01 s⁻¹ of less than or equal to 700 Pa·s, preferably less than or equal to 500 Pa·s, more preferably less than or equal to 400 Pa·s, and even more preferably less than or equal to 300 Pa·s, according to the Rheology Test Method.
I. The composition according to Paragraphs A-H, comprising a time weighted force area of from about 1×10⁴ g to about 2.2×10⁴ g, preferably from about 1.1×10⁴ g to about 2.0×10⁴ g, more preferably from about 1.2×10⁴ g to about 1.9×10⁴ g, and even more preferably from about 1.3×10⁴ g to about 1.7×10⁴ g, according to the Tack Method.
J. The composition according to Paragraphs A-I, comprising a time weighted force area of less than 2.3×10⁴ g, preferably less than 2.1×10⁴ g, more preferably less than 1.9×10⁴ g, and even more preferably less than 1.8×10⁴ g, according to the Tack Method.
K. The composition according to Paragraphs A-J, wherein the polymer system is free of or substantially free of xanthan gum and hydroxyethylcellulose.
L. The composition according to Paragraphs A-K, wherein the composition is substantially free of emulsifiers other than the emulsifying properties of the microcrystalline cellulose, Sphingomonas ferment extract, and cellulose gum.
M. The composition according to Paragraphs A-L, comprising:
   a. a weight ratio of microcrystalline cellulose to Sphingomonas ferment extract that is greater than or equal to 1:1;
   b. a weight ratio of Sphingomonas ferment extract to cellulose gum that is greater than or equal to 1:1; and
   c. a weight ratio of microcrystalline cellulose to cellulose gum that is greater than or equal to 1:1.
N. The composition according to Paragraphs A-M, wherein the composition comprises from about 0.50% to about 0.95%, preferably from about 0.60% to about 0.85%, more preferably from about 0.62% to about 0.85%, and even more preferably from about 0.65% to about 0.80% of the polymer system.
O. The composition according to Paragraphs A-N, wherein the composition is clean.
P. The composition according to Paragraphs A-O, wherein the composition comprises less than 10% silicone elastomer, preferably less than 7% silicone elastomer, more preferably less than 5% silicone elastomer, and even more preferably less than 1% silicone elastomer.
Q. The composition according to Paragraphs A-P, wherein the composition comprises less than 1% snow mushroom (Tremella fuciformis) extract, preferably less than 0.5% snow mushroom extract, more preferably less than or equal to 0.1% snow mushroom extract, and even more preferably the composition can be free of or substantially free of snow mushroom extract.
R. The composition according to Paragraphs A-Q, wherein the composition comprises less than 0.5% sodium hyaluronate, preferably less than 0.3% sodium hyaluronate, preferably less than 0.1% sodium hyaluronate, and even more preferably the composition can be free of or substantially free of sodium hyaluronate.
S. A serum product comprising:
   a. a bottle containing the serum composition according to Paragraphs A-R;
   b. a dropper coupled to the bottle.
T. The composition according to Paragraphs A-S, wherein the composition is a single phase, as determined by visual detection.
U. The composition according to Paragraphs A-T, wherein the composition comprises no visible particles, as determined by visual detection.
V. The composition according to Paragraphs A-U, wherein the microcrystalline cellulose particles comprise a diameter of from about 5 μm to about 20 μm, preferably from about 10 μm to about 20 μm, more preferably from about 10 μm to about 30 μm, and even more preferably from about 10 μm to about 40 μm.
W. Use of a polymer system comprising microcrystalline cellulose, Sphingomonas ferment extract, and cellulose gum in a skin care serum composition according to Paragraphs A-R and T-V for providing silky feel during and/or after application.
X. Use of a polymer system comprising microcrystalline cellulose, Sphingomonas ferment extract, and cellulose gum in a skin care serum composition according to Paragraphs A-R and T-V for providing physical stability of the composition.
Y. The use of the polymer system of Paragraph X, wherein physical stability of the composition is assessed by a visual detection and wherein there is no phase separation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A physically stable, uniform, skin care serum composition, wherein the composition is an emulsion comprising:
a. a continuous phase comprising a polymer system comprising microcrystalline cellulose, Sphingomonas ferment extract, and cellulose gum; a liquid carrier; and a skin care active;
b. a dispersed phase comprising an emollient.

2. The composition of claim 1, wherein the skin care active is chosen from vitamin E, vitamin C, retinol, undecylenoyl phenylalanine, niacinamide, botanicals, pre-, pro- or post-biotics, and/or derivatives and mixtures thereof.

3. The composition of claim 2, wherein the skin care active comprises a retinol and/or derivatives thereof.

4. The composition of 3, wherein the composition comprises greater than or equal to 75% of the retinol and/or derivatives thereof remaining as compared to the amount of retinol and/or derivatives thereof at the time of manufacture according to the HPLC Method.

5. The composition of 3, wherein the composition comprises greater than or equal to 85% of the retinol and/or derivatives thereof remaining as compared to the amount of retinol and/or derivatives thereof at the time of manufacture according to the HPLC Method.

6. The composition of claim 1, comprising:
a. a viscosity of less than or equal to 300 Pa·s at 0.01 s⁻¹ according to the Rheology Test Method; and
b. a viscosity of less than or equal to 0.6 Pa·s at 20 s⁻¹ according to the Rheology Test Method.

7. The composition of claim 6, wherein the composition comprises a slope between 0.01 s⁻¹ and 0.1 s⁻¹ shear rate greater than −600 and less than 0.

8. The composition of claim 1, comprising a viscosity at 0.01 s⁻¹ of less than or equal to 700 Pa·s.

9. The composition of claim 1, comprising a time weighted force area of less than 2.3×10⁴ g.

10. The composition of claim 1, wherein the polymer system is free of or substantially free of xanthan gum and hydroxyethylcellulose.

11. The skin care composition of claim 1, wherein the composition is substantially free of emulsifiers other than the emulsifying properties of the microcrystalline cellulose, Sphingomonas ferment extract, and cellulose gum.

12. The composition of claim 1, comprising:
a. a weight ratio of microcrystalline cellulose to Sphingomonas ferment extract that is greater than or equal to 1:1;
b. a weight ratio of Sphingomonas ferment extract to cellulose gum that is greater than or equal to 1:1; and
c. a weight ratio of microcrystalline cellulose to cellulose gum that is greater than or equal to 1:1.

13. The composition of claim 1, wherein the composition comprises from about 0.50% to about 0.95% of the polymer system.

14. The skin care composition of claim 1, wherein the composition is clean.

15. A serum product comprising:
a. a bottle containing the serum composition of claim 1;
b. a dropper coupled to the bottle.

16. The composition of claim 1, wherein the composition comprises no visible particles.

17. The composition of claim 1 wherein the microcrystalline cellulose particles comprise a diameter of from about 5 μm to about 20 μm.

18. A physically stable, uniform, skin care serum composition comprising:
a. a polymer system comprising microcrystalline cellulose, Sphingomonas ferment extract, and cellulose gum;
b. a liquid carrier comprising water; and
c. a skin care active;
wherein the composition does not comprise an emulsion.

19. The composition of claim 18, wherein the skin care active is chosen from wherein the skin care active is chosen from vitamin E, vitamin C, retinol, undecylenoyl phenylalanine, niacinamide, botanicals, pre-, pro- or post-biotics, and/or derivatives and mixtures thereof.
